(19) United States
(12) Patent Application Publication
(10) Pub. No.: US 2024/0191194 A1
(43) Pub. Date: Jun. 13, 2024

(54) METHOD FOR PRODUCING CULTURE CELLS REQUIRING A SUPPLY OF FERRIC IRON

(71) Applicant: Erypharm, Paris (FR)

(72) Inventors: Guillaume ROUSSEAU, Paris (FR); Marie-Catherine GIARRATANA, Saint-Ouen (FR); Florent MATHIEU, Saint-Germain-en-Laye (FR)

(21) Appl. No.: 18/551,317

(22) PCT Filed: Mar. 18, 2022
(86) PCT No.: PCT/EP2022/057221

§ 371 Date: Sep. 19, 2023

(30) Foreign Application Priority Data
Mar. 19, 2021 (FR) FR2102786

(51) Int. Cl. C12N 5/078; C12M 1/00; C12N 5/00

(52) U.S. Cl. CPC C12N 5/0641; C12M 29/04; C12M 29/10; C12M 29/16; C12N 5/0018; C12N 2500/24; C12N 2501/10; C12N 2501/20; C12N 2501/30

(57) ABSTRACT

The present invention relates to a method for producing cultured cells requiring a supply of ferric iron, comprising a step of culturing cells to be cultured in a perfusion bioreactor containing a culture medium comprising transferrin, wherein the bioreactor is supplied with a source of ferric iron and wherein the culture medium is filtered at the bioreactor outlet by a filter having a cut-off threshold of less than 76 kDa.

### FIELD OF THE INVENTION

The present invention relates to a process for producing cultured cells requiring ferric iron.

### TECHNICAL BACKGROUND

Some technologies for replacing products of animal or human origin, such as cultured meat, cultured erythroid or red blood cells, require the production of large quantities of animal or human cells.

However, industrial-scale production of animal or human cells faces a number of obstacles, not least the fact that costs are too high and yields too low.

Most of the costs come from proteins, hormones and growth factors, particularly recombinant ones, added to the culture medium to replace or supplement serum.

By way of example, it has been calculated that for a batch culture of cells destined for the production of cultured meat, the cost of the four required growth factors, i.e. insulin, transferrin, FGF-2 and TGF-β, represented 99% of the cost of the culture medium, with 96% for FGF-2 and TGF-β alone (Specht (2020) “An analysis of culture medium costs and production volumes for cultured meat”, The Good Food Institute). However, transferrin-related costs are higher for ferric iron-intensive cultures, such as red blood cell cultures.

Thus, Giarratana et al (2011) “Proof of principle for transfusion of in vitro-generated red blood cells”, Blood 118:5071-5079 describe the ex vivo production of cultured red blood cells from hematopoietic stem cells isolated from peripheral blood. However, the process used is too expensive for industrial or medical application, in particular because it requires completely renewing, several times a week, the culture medium, which is rich in growth factors and transferrin (330 μg/mL), i.e. around 30 times more transferrin in concentration than would be required for the production of cells for the preparation of cultured meat in the above example.

Non-protein substitutes for transferrin have been proposed, such as ferric citrate (Eto et al. (1991) Agric. Biol. Chem. 55:863-865).

However, the industrial feasibility of this type of substitution has not yet been established, particularly for ferric iron-intensive cultures.

There is therefore a need for an industrializable process for producing cultured cells requiring ferric iron.

### SUMMARY OF THE INVENTION

The present invention stems from the inventors' unexpected finding that it was possible to maximize cell production in a perfusion bioreactor while minimizing the amount of protein, in particular of transferrin, required.

Thus, the present invention relates to a method for producing cultured cells requiring a supply of ferric iron, comprising a step of culturing cells to be cultured in a perfusion bioreactor containing a culture medium comprising transferrin, in which the bioreactor is supplied with a source of ferric iron and in which the culture medium is filtered at the bioreactor outlet by a filter having a cut-off threshold of less than 76 kDa.

Advantageously, the process defined above makes it possible to increase the ratio of quantity of cells produced/quantity of transferrin used compared with methods of the prior art.

### DETAILED DESCRIPTION OF THE INVENTION

As a preliminary point, it is reminded that the term “comprising” means “including”, “containing” or “encompassing”, i.e. when an object “comprises” an element or several elements, elements other than those mentioned can also be included in the object. In contrast, the expression “consisting of” means “constituted by”, i.e. when an object “consists of” an element or several elements, the object cannot include elements other than those mentioned.

### Cells

Cells according to the invention are of any type requiring a supply of ferric iron.

Preferably, they are eukaryotic cells, more preferably animal cells, notably bird, mammal or human cells.

They can be cells to be cultivated for themselves, such as NK cells, lymphocytes, in particular chimeric antigen receptor T cells (CAR T cells), erythroid cells, in particular erythroblasts, cultured red blood cells or cultured meat cells, or cells to be cultivated for producing molecules of interest, in particular proteins, more particularly antibodies or antibody derivatives, notably monoclonal antibodies.

Preferably, the cultured cells requiring ferric iron are cells containing hemoglobin and/or myoglobin.

Preferably, the cultured cells requiring ferric iron are erythroid cells, in particular erythroblasts, cultured red blood cells or cultured meat cells.

As is understood herein, “cultured meat” is synonymous with “synthetic meat” or “clean meat”.

Cells according to the invention may be stem cells, progenitor cells, or cells of an immortalized cell line of the erythroid lineage.

The stem cells may be embryonic stem cells (ESC), induced pluripotent stem cells (iPSC), or hematopoietic stem and/or progenitor cells (HSC/HP). Preferably, the method according to the invention uses hematopoietic stem cells (HSC) as the cell source.

Cells from an immortalized cell line of the erythroid lineage can be immortalized at the erythroid progenitor or erythroid precursor stage. Hematopoietic stem cells (HSC) can also be immortalized.

Immortalization is preferably carried out conditionally. These immortalized cells can then be passed indefinitely in vitro, cryopreserved and recovered, and conditionally produce fully differentiated red blood cells from a defined and well-characterized source. Conditional immortalization can be achieved by any method well known to those skilled in the art.

Embryonic stem cells (ESC) and induced pluripotent stem cells (iPSC) are pluripotent stem cells. These cells are capable of both differentiating into numerous cell types and self-replicating. They can maintain this pluripotent differentiation while multiplying by division. Embryonic stem cells refer to pluripotent stem cells derived from embryos at the blastocyst stage, the early stage of animal development. Induced pluripotent stem cells (iPSCs) are produced by introducing several types of transcription factor genes into somatic cells such as fibroblasts.

Embryonic stem cells (ESC) according to the invention are obtained by any means not requiring the destruction of human embryos. For example, using the technology described by Chung et al (Chung et al, Human Embryonic Stem Cell lines generated without embryo destruction, Cell Stem Cell (2008)). Furthermore, the method according to the invention does not use human embryos and is not intended to induce the development process of a human being.

According to an embodiment of the invention, said stem cells used in the method according to the invention are not human embryonic stem cells (hESC) and/or iPSCs.

The hematopoietic stem cells (HSC) used in the method according to the invention are multipotent cells. They are capable of differentiating into all blood cell differentiation lineages, and of self-replicating while maintaining their multipotency.

Cells of an immortalized erythroid lineage are cells already committed to the erythroid lineage but capable of self-replicating and under external control of differentiating into erythroid lineage cells.

The hematopoietic stem and/or progenitor cells (HSC/HP) used in the method according to the invention can be derived from any source, including bone marrow, umbilical cord/placental blood or peripheral blood, with or without prior mobilization.

The origin of stem cells and cells of an immortalized cell lineage of the erythroid lineage is not particularly limited as long as it is derived from a mammal. Preferred examples include humans, dogs, cats, mice, rats, rabbits, pigs, cows, horses, sheep, goats and the like, with humans being most preferred.

The cells used in the method according to the invention can produce, without limitation, red blood cells from universal donors, red blood cells from a rare blood group, red blood cells for personalized medicine (for example, autologous transfusion, possibly with genetic engineering) and red blood cells designed to include one or more proteins of interest.

In certain embodiments which may be combined with any of the preceding embodiments, said cells used in the method according to the invention may be isolated from a patient having a rare blood group comprising, without limitation, Oh, CDE/CDE, CdE/CdE, CwD−/CwD−, −D−/−D−, Rhnull, Rh: −51, LW (a−b+), LW (ab−), SsU−, SsU (+), pp, Pk, Lu (a+b−), Lu (ab−), Kp (a+b−), Kp (ab−), Js (a+b−), Ko, K: −11, Fy (ab−), Jk (ab−), Di(b−), I−, Yt (a−), Sc: −1, Co (a−), Co (ab−), Do (a−), Vel−, Ge−, Lan−, Lan (+), Gy (a−), Hy−, At (a−), Jr (a−), In (b−), Tc (a−), Cr (a−), Er (a−), Ok (a−), JMH− and En (a−).

According to one embodiment of the invention, said cells can be embryonic stem cells (ESC), preferably human (hESC) and preferably selected from the group consisting of H1, H9, HUES-1, HUES-2, HUES-3, HUES-7, CLO1 lines and pluripotent stem cells (iPSC), preferably human (hiPSC).

Preferably, said cells are hematopoietic stem cells (HSC), more preferably human.

In the case of cells derived from umbilical cord/placental blood or peripheral blood, bone marrow or apheresis, a specific CD34+ cell selection step can be carried out prior to step a) of the method according to the invention.

Apheresis is a technique for removing certain blood components using extracorporeal blood circulation. The components to be removed are separated by centrifugation and extracted, while those not removed are reinjected into the donor (blood) or patient (therapeutic apheresis).

CD34+ (positive) means that the CD (differentiation cluster) 34 antigen is expressed on the cell surface. This antigen is a marker of hematopoietic stem cells and hematopoietic progenitor cells, and disappears as they differentiate. Similar cell populations also include CD133-positive cells.

In cases where the starting cells are ESCs, iPSCs or cells of an immortalized cell line of the erythroid lineage, pre-culture steps can be added upstream of the culture step in the bioreactor to multiply the cells and possibly commit them to a differentiation pathway, notably of the erythroid lineage.

Preferably, the cultured cells requiring a supply of ferric iron are cultured red blood cells, and the cells to be cultured are erythroid stem cells or progenitor cells, or cells of an immortalized cell line of the erythroid lineage.

Whatever the cell source, a preliminary freezing step of the cells to be cultured is often required for transport and preservation reasons. Cell freezing methods are well known in the state of the art, and include programmed temperature descent and the use of cryoprotectants such as lactose or dimethyl sulfoxide (DMSO). When added to the medium, DMSO prevents the formation of intracellular and extracellular crystals in the cells during the freezing process.

Thus, in a particular embodiment of the invention, the method according to the invention comprises a cell thawing step, prior to the culture step in a perfusion bioreactor, in the case where the cells to be cultured are frozen. Cell thawing methods are well known to the person of skill in the art.

Thawing is an important step in the process, particularly when DMSO has been used for freezing. This compound is cryopreservative as long as the cell suspension is kept in liquid nitrogen or nitrogen vapor. However, it becomes cytotoxic as soon as the cell suspension is thawed. DMSO therefore needs to be removed very quickly by several washing steps as soon as the cells are thawed, as is well known to the person of skill in the art.

In other cases, the starting cells may be fresh, i.e. the time between cell collection and culture is short enough not to require freezing, preferably less than 48 hours. This may be the case, for example, when the sampling center is located on the same site or close to the production center.

### Culture Method

Perfusion is a continuous culture method in which cells are retained in the bioreactor, or circulated and returned to the bioreactor, while used culture medium is evacuated, compensated by the addition of a perfusion liquid to renew the culture medium. The used and discharged culture medium therefore contains no cells. In this case, the culture medium is filtered at the bioreactor outlet to produce a permeate.

The aim of the culture stage in a perfusion bioreactor according to the invention is to multiply the cultured cells and, in the case of the production of cultured red blood cells, to complete their differentiation to an enucleated reticulocyte stage.

The culture is carried out in a bioreactor suitable for perfusion culture. Numerous models of bioreactors suitable for perfusion cell culture are known to the person skilled in the art.

The bioreactor preferably has a capacity of from 0.5 to 5000 L.

Preferably, the bioreactor comprises a gas exchange means to satisfy the oxygen requirements of the cells and control the pH by controlling the supply and/or removal of carbon dioxide (CO₂). Preferably, the gas exchange means is low shear.

Preferably, at least one of the following growing conditions, more preferably all of them, are controlled or regulated:
Stirring;
pH;
Dissolved oxygen (DO);
Temperature;
The volume or level of the bioreactor;
Perfusion rate;
Supply in nutrients, in particular selected from carbohydrates, amino acids, vitamins and iron;
Supply in growth factors, cytokines and/or hormones;
Fouling of the bioreactor and clogging of filtering elements.

Preferably, the culture is carried out for a period of time sufficient to obtain a cell concentration greater than 30 million cells/ml. Preferably this period of time is from 5 days to 25 days, more preferably from 10 days to 20 days.

Preferably, the culture temperature is between 33° C. and 40° C., more preferably between 35° C. and 39° C., and even more preferably between 36° C. and 38° C.

Preferably, the culture pH is between 7 and 8, more preferably between 7.2 and 7.7.

Preferably, the culture DO is between 1% and 100%, more preferably between 10% and 100%.

Advantageously, the perfusion bioreactor culture step allows cultured cells to be concentrated to levels unattainable in batch and fed-batch culture, i.e. above 30 million cells/ml and up to 200 million cells/ml. Advantageously, the perfusion bioreactor culture step of the process of the invention can also be used to differentiate the cultured cells. Advantageously, in the case of the production of cultured red blood cells, the rate of enucleated cells at the end of the perfusion bioreactor culture stage exceeds 50%, 60%, 70% or 80%.

In one embodiment of the invention, the perfusion culture step is preceded by at least one batch or fed-batch bioreactor culture step.

In batch cultures, the medium is not renewed, so the cells have only a limited supply of nutrients. Fed-batch culture, in contrast, corresponds to a batch culture with a supply of nutrients and/or culture medium.

The advantage of the batch or fed-batch bioreactor culture step(s) is that the cells to be cultured are pre-amplified and, in the case of the production of cultured red blood cells, to commit or differentiate the starting cells, or to enhance their commitment or their differentiation, to/in the erythroid lineage.

Thus, in the case of the production of cultured red blood cells, it is possible, in an embodiment of the invention, to continue the culture step in a batch or fed-batch bioreactor until the cultured cells are committed to the erythroid lineage. According to this embodiment of the invention, cells are considered to be sufficiently committed to the erythroid lineage when they display one or more characteristics specific to the erythroid lineage, such as a percentage of cells displaying the CD235 marker, measurable for example by flow cytometry, greater than 50%, or a percentage of cells with an erythroid phenotype, which can be measured for example by cytological counting after staining with the May-Grünwald Giemsa dye, greater than 50%.

One or more successive or iterative cultures in a batch or fed-batch bioreactor can be carried out, for example between 1 and 4 times.

The batch or fed-batch bioreactor type is not particularly limited as long as it can generally culture animal cells. Preferably, the bioreactor of step a) has a capacity of 0.5 to 5000 L, more preferably 0.5 to 500 L.

In an embodiment of the invention, the process for producing cultured cells according to the invention comprises a step for purifying the cultured cells obtained after the perfusion bioreactor culture step.

The purpose of the purification step is:
to wash the cells to eliminate potentially toxic residues from the process; and
in the case of production of cultured red blood cells, to sort the cells in order to concentrate enucleated cells as much as possible.

The purification step may comprise one or more operations, in particular a particle sorting operation and a washing operation. The washing operation may be performed either before and/or after the particle sorting operation.

In the case of cultured red blood cell production, particle sorting increases the enucleated cell rate, in particular by eliminating erythroblasts and potential residual myeloid cells. Erythroblasts are cultured cells that have not reached the enucleated cell differentiation stage, i.e. into reticulocytes or red blood cells. Particle sorting also eliminates cellular waste, such as cell debris, DNA and pyrenocytes.

Particle sorting according to the invention can comprise at least one operation selected from the group consisting of tangential filtration, frontal filtration and elutriation.

Tangential-flow filtration is well known to the person skilled in the art. It is a filtration method that separates particles from a liquid according to their size. In tangential filtration, the liquid flow is parallel to the filter, in contrast to dead-end filtration, in which the liquid flow is perpendicular to the filter. It is the pressure of the fluid that allows it to pass through the filter. As a result, smaller particles pass through the filter, while larger ones continue their journey via the liquid flow.

Dead-end filtration is well known to the person skilled in the art. Its principle consists in retaining the particles to be eliminated inside a porous network that forms the filter. Filtration is based on 4 mechanisms: (i) particle/wall adhesion forces, (ii) inter-particle adhesion forces, (iii) steric hindrance and (iv) the drag force of the fluid on the particles. Its effectiveness depends in particular on the material, pore size, type of fiber entanglement and the ratio of filtration surface area to the quantity of material to be filtered.

Elutriation is a technique for the separation and particle size analysis of particles of different sizes. Elutriation is based on Stokes' law. A fluid containing cells is sent at a known speed into a chamber where the particles are subjected to a controlled centrifugal force. The particles remain in suspension when the two forces (fluid drag and centrifugal force) cancel each other out.

Preferably, the particle sorting operation according to the invention comprises a succession of frontal filtrations and possibly elutriation.

The purpose of the washing operation is in particular to reduce the quantities of toxic compounds potentially present in the cell culture of step b) below their toxicity threshold.

The washing operation may comprise one or more centrifugations and/or one or more elutriations.

Centrifugation is well known to the person skilled in the art. It is a method for separating compounds in a mixture on the basis of their density difference and their drag, by subjecting them to unidirectional centrifugal force and possibly to an opposing flow.

Preferably, the washing step according to the invention comprises a succession of elutriation operations.

The particle sorting, washing and formulation steps are carried out in a period of less than 72 hours, and more preferably less than 12 hours.

### Culture Medium

Preferably, the bioreactor is supplied with a perfusion liquid, which may comprise a culture medium.

The person skilled in the art is able to select or prepare a suitable culture medium according to the invention. Examples of suitable culture media are those described in the International publication WO2011/101468A1 and in the article Giarratana et al. (2011) “Proof of principle for transfusion of in vitro-generated red blood cells”, Blood 118:5071-5079.

The culture medium generally comprises a basal culture medium for eukaryotic cells, such as a DMEM, IMDM, RPMI 1640, MEM or DMEM/F12 medium, which are well known to the person skilled in the art and widely available commercially.

The culture medium or perfusion liquid may also include plasma, in particular in an amount of 0.5% to 6% (v/v).

Preferably, the culture medium or perfusion liquid also includes nutrients and growth factors, cytokines and/or hormones.

Thus, the person skilled in the art is able to adapt the culture medium and the perfusion liquid by adding certain components or modulating the quantities of certain components, including sodium, potassium, calcium, magnesium, phosphorus, chlorine, various amino acids, various nucleosides, various vitamins, various antioxidants, fatty acids, carbohydrates and analogues, fetal bovine serum, human plasma, human serum, horse serum, heparin, cholesterol, ethanolamine, sodium selenite, monothioglycerol, mercaptoethanol, bovine serum albumin, human serum albumin, sodium pyruvate, polyethylene glycol, poloxamers, surfactants, lipid droplets, antibiotics agar, collagen, methylcellulose, various cytokines, various hormones, various growth factors, various small molecules, various extracellular matrices and various cell adhesion molecules.

Examples of cytokines included in the culture medium or the perfusion liquid include interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-18 (IL-18)), Interleukin-21 (IL-21), interferon-A (IFN-α), interferon-3 (IFN-β), interferon-γ (IFN-γ), granulocyte colony-stimulating factor (G-CSF), monocyte colony-stimulating factor (M-CSF), granulocyte-macrophage colony-stimulating factor (GM-CSF), stem cell factor (SCF), flk2/flt3 ligand (FL), leukemic cell inhibitory factor (LIF), oncostatin M (OM), erythropoietin (EPO), thrombopoietin (TPO) However, it is not limited to these.

The various small molecules included in the culture medium or the perfusion liquid may include aryl hydrocarbon receptor antagonists such as StemRegenin1 (SR1), hematopoietic stem cell self-renewal agonists such as UM171, and the like, but are not limited to these.

Growth factors included in the culture medium or the perfusion liquid may include transforming growth factor-a (TGF-a), transforming growth factor-β (TGF-β), macrophage inflammatory protein-la (MIP-1a), epidermal growth factor (EGF), fibroblast growth factor-1, 2, 3, 4, 5, 6, 7, 8 or 9 (FGF-1, 2, 3, 4, 5, 6, 7, 8, 9), nerve cell growth factor (NGF), vascular endothelial growth factor (VEGF), hepatocyte growth factor (HGF), leukemia inhibitory factor (LIF), nexin I protease, nexin II protease, platelet-derived growth factor (PDGF), cholinergic differentiation factor (CDF), various chemokines, Notch ligands (such as Delta1), Wnt proteins, angiopoietin-like proteins 2, 3, 5 or 7 (Angpt 2, 3, 5, 7), insulin-like growth factors (GF), insulin-like growth factor binding protein (IGFBP), pleiotrophin, and the like, but are not limited to these.

Hormones in the culture medium or perfusion liquid may include hormones from the glucocorticoid family, such as dexamethasone or hydrocortisone, from the thyroid hormone family, such as T3 and T4, ACTH, alpha-MSH or insulin.

### Filter

The filter according to the invention removes the used culture medium in the form of a permeate, while preserving the cells grown in the bioreactor.

The “cut-off” value, or filter pore size, is defined as the molar mass of the smallest compound in the filtered medium which retention by the filter is 90%. The cut-off point is generally specified for commercial filters.

Preferably, the cut-off according to the invention is less than 50 kDa, preferably less than 15 kDa. Preferably, the cut-off according to the invention is greater than 1 kDa. Preferably, the cut-off according to the invention is from 1 kDa to 50 kDa, more preferably from 1 kDa to 15 kDa.

Preferably, the filter is a tangential filtration system.

Tangential-flow filtration is well known to the person skilled in the art. It is a filtration method that separates particles from a liquid according to their size. In tangential filtration, the liquid flow is parallel to the filter, in contrast to dead-end filtration, in which the liquid flow is perpendicular to the filter. It is the pressure of the fluid that allows it to pass through the filter. As a result, smaller particles pass through the filter, while larger ones continue their journey via the liquid flow.

Preferably, the filter consists of hollow fibers or of a filter cassette.

### Source of Ferric Iron

The ferric iron source can supply the bioreactor directly, via a dedicated supply line, or via the perfusion liquid. In the latter case, the ferric iron source is included in the perfusion liquid.

Preferably, the ferric iron source is not proteinaceous in nature, i.e. it is aproteic. More preferably, the ferric iron source is not transferrin.

Preferably, the ferric iron source is a ferric iron salt or a ferric iron complex.

Examples of ferric salts include Fe chloride, iron nitrate, iron sulfate or iron disphosphate.

More preferably, the ferric iron source is a complex of ferric iron and a chelating agent.

Preferably, the chelating agent is selected from the group consisting of citric acid, methylglycinediacetic acid (MGDA, eg. Trilon® M), 2,4-pentanedione (ACAC), N-(2-aminoethyl)iminodiacetic acid (AEIDA), 1,2-dihydroxybenzene (CAT), 1,2-diaminocyclohexanetetraacetic acid (CDTA), acethydroxamic acid, acetic acid, desferriferrioxamine-B (DFB), 1,8-dihydroxynaphthalene-4-sulfonic acid (DHNS), dipicolinic acid (DIPIC), 1-2-dimethylethylenediaminetetraacetic acid (DMEDTA), 1,4,7,10-tetraazacyclododecane-N,N′,N″,N-″′-tetraacetic acid (DOTA), diethylenetriaminepentaacetic acid (DTPA), ethylenediaminetetraacetic acid (EDTA), oxybis(ethylenenitrilo)tetraacetic acid (EEDTA), ethylenebis(oxyethylenitrilo)tetraacetic acid (EGTA), ethylene-N,N′-bis(2-hydroxyphenylglycine (EHPG), glycine (Gly), N,N′-Bis(2-hydroxybenzyl)ethylenediamine-N,N′-diacetic acid (HBED), N-(2-hydroxybenzyl)ethylenediamine-N,N′,N-triacetic acid (HBET), N-(2-hydroxyethyl)ethylenediamine-N,N′,N′-triacetic acid (HEDTA), N-(2-hydroxyethyl)iminodiacetic acid (HIDA), iminodiacetic acid, kojic acid, nitrilotriacetic acid (NTA), oxalic acid, propylenediaminetetraacetic acid (PDTA), picolinic acid (PIC), N,N′-bis(2-methyl-3-hydroxy-5-hydroxymethyl-4-pyridylmethyl)ethylenediamine-N,N′-diacetic acid (PLED), 1,4,8,11-tetraazacyclotetradecane-N,N′,N″,N″′-tetraacetic acid (TETA), 3,5-disulfocatechol (Tiron), trimethylenediaminetetraacetic acid (TMDTA), 1,4,7,10-tetraazacyclotridecane-N,N′,N″,N″′-tetraacetic acid (TRITA), triethylenetetraminehexaacetic acid (TTHA), deferoxamine, deferiprone, tropolone and hinokitiol

More preferably, the ferric iron source is a complex of ferric iron and citrate.

Preferably, the ferric iron source is supplied to the bioreactor or the perfusion liquid in a quantity sufficient to maintain the saturation coefficient of transferrin contained in the culture medium at a value greater than 10%, preferably greater than 50%.

### Transferrin

The transferrin according to the invention can be of any type capable of supplying ferric iron to cells in culture.

Preferably, the transferrin is a bird, animal, especially mammalian, e.g. bovine, porcine or human, transferrin. Preferably, the transferrin belongs to the same species as that of the cultured cells. The transferrin can be extracted from plasma. Preferably, the transferrin is recombinant. Preferably, the transferrin is a human transferrin produced recombinantly in rice.

Preferably, the transferrin concentration in the bioreactor is of from 10 to 3,000 μg/ml, more preferably of from 10 to 500 μg/ml.

Preferably, transferrin is loaded with ferric iron before being added to the bioreactor or culture medium.

Preferably, the transferrin saturation coefficient is maintained at a value greater than 10%, preferably greater than 50%.

The transferrin saturation coefficient can be measured spectrophotometrically. in particular as described by Bates & Schlabach (1973) J. Biol. Chem. 248:3228-3232 or by Steere et al. (2012) J. Inorg. Biochem. 116:37-44.

The transferrin saturation coefficient can be controlled by modulating the ferric iron source supply in the bioreactor or in the perfusion liquid.

Preferably, the method according to the invention consumes less than 10⁻¹⁰ g, more preferably less than 10⁻¹¹ g, even more preferably less than 5.10⁻¹² g of transferrin per cultured cell, in particular per cultured red blood cell, produced.

The invention will be further explained with the aid of the following non-limiting Example.

### Example

The cell production method according to the invention is compared with a successive batch culture (comparative example) based on the article by Giarratana et al. (2011) “Proof of principle for transfusion of in vitro-generated red blood cells”, Blood 118:5071-5079, both sized to produce the equivalent of one blood bag, i.e. 2.10¹² red blood cells.

In the case of batch culture, the entire culture medium must be renewed twice a week, and the volume increased.

The main quantities are detailed in the table below:

TABLE 1
Batch culture (comparative example)

| | D0 | D3 | D7 | D10 | D14 | D17 |
|---|---|---|---|---|---|---|
| [Cells]_end (cells/mL) | NA | 1.0E+06 | 1.0E+06 | 2.0E+06 | 2.5E+06 | 2.5E+06 |
| [Cells]_start (cells/mL) | 5.0E+04 | 5.0E+04 | 4.0E+05 | 2.0E+06 | 2.5E+06 | NA |
| Enucleation rate | 0% | <5% | 6% | 33% | 57% | 80% |
| Expansion factor/phase | NA | 20 | 20 | 5 | 1.25 | 1 |
| Cumulative expansion | | | | | | 2500 |
| factor | | | | | | |
| Volume required/phase (L) | 0.5 | 10.0 | 200.0 | 1000.0 | 1000.0 | NA |

The total volume of medium required is 2210.5 L. Transferrin is contained in the medium at 300 μg/mL. The total amount of transferrin required is 663.15 g.

By comparison, in the case of a perfusion culture according to the invention (25 L bioreactor with a hollow-fiber filter with a 10 kDa cut-off at the outlet) in which transferrin is continuously recharged by a source of ferric iron (iron citrate), transferrin is only supplied to the culture medium used to fill the reactor at the start of the method, the composition of is similar to that of the culture medium in successive batches.

The main quantities are detailed in the table below:

TABLE 2
Perfusion culture according to the invention

| | D0 | D17 |
|---|---|---|
| [Cells]_end (cells/mL) | NA | 1.0E+08 |
| [Cells]_start (cells/mL) | 4.0E+04 | NA |
| Enucleation rate | 0% | 80% |
| Cumulative expansion factor | | 2500 |
| Volume required (L) | 25 | |

Transferrin is contained in the culture medium occupying the bioreactor at a concentration of 300 μ/mL. The total amount of transferrin required is 7.5 g.

It can be seen that the method according to the invention reduces the quantity of transferrin required by a factor of 88.4, for the same quantity of cells produced and the same expansion factor. Moreover, the quantity of transferrin relative to the quantity of red blood cells produced is 3.75.10⁻¹² g/red blood cell.

1. A method for producing cultured cells requiring a supply of ferric iron, comprising a step of culturing cells to be cultured in a perfusion bioreactor containing a culture medium comprising transferrin, wherein the bioreactor is supplied with a source of ferric iron and wherein the culture medium is filtered at the bioreactor outlet by a filter having a cut-off value of less than 76 kDa.

2. The method according to claim 1, wherein the cultured cells requiring a ferric iron supply are cells which contain hemoglobin and/or myoglobin.

3. The method according to claim 1, wherein the cultured cells requiring a ferric iron supply are erythroid cells, cultured red blood cells or cultured meat cells.

4. The method according to claim 1, wherein the cultured cells requiring a ferric iron supply are cultured red blood cells and the cells to be cultured are erythroid stem or progenitor cells or cells of an immortalized cell line of the erythroid lineage.

5. The method according to claim 1, wherein a perfusion liquid and/or the culture medium further comprises nutrients as well as growth factors, cytokines and/or hormones.

6. The method according to claim 1, wherein the cut-off is less than 50 kDa.

7. The method according to claim 1, wherein the ferric iron source is a ferric iron salt or a ferric iron complex.

8. The method according to claim 1, wherein the ferric iron source is a complex of ferric iron and a chelating agent.

9. The method according to claim 1, wherein the ferric iron source is a complex of ferric iron and citrate.

10. The method according to claim 1, wherein the bioreactor filter is a tangential filtration system.

11. The method according to claim 1, wherein the bioreactor filter consists of hollow fibers.

12. The method according to claim 1, wherein the transferrin saturation coefficient is maintained at a value greater than 10%.

13. The method according to claim 1, wherein the transferrin concentration in the bioreactor is of from 10 to 3,000 μg/ml.

14. The method according to claim 1, wherein the cut-off is less than 15 kDa.

15. The method according to claim 1, wherein the transferrin saturation coefficient is maintained at a value greater than 50%.
